(19) United States
(12) Patent Application Publication
(10) Pub. No.: US 2011/0172321 A1
(43) Pub. Date: Jul. 14, 2011

(54) HOTMELT VISCOSITY STABILIZER

(75) Inventors: Wilhelm Laufer, Ellerstadt (DE); Bernd Kray, Speyer (DE); Peter Schuster, Altrip (DE); Christian Scheffner, Sandhausen (DE); Serdar Uestuenbas, Mannheim (DE); Marc Leimenstoll, Dormagen (DE); Peter Reichert, Dormagen (DE); Eduard Mayer, Dormagen (DE); Matthias Wintermantel, Bergisch Gladbach (DE)

(73) Assignee: RHEIN CHEMIE RHEINAU GmbH, Mannheim (DE)

(21) Appl. No.: 12/898,853

(22) Filed: Oct. 6, 2010

(30) Foreign Application Priority Data
Oct. 16, 2009 (EP) 09173303.0

(51) Int. Cl. C08K 5/29; C08L 75/06; C08J 9/35; C09D 175/06; C07C 267/00

(52) U.S. Cl. 521/94; 524/195; 564/252; 564/5

(57) ABSTRACT

The invention relates to innovative viscosity stabilizers for hotmelts, to melt-viscosity-stable hotmelts comprising these viscosity stabilizers, to a process for preparing them and to their use.

The invention relates to innovative viscosity stabilizers for hotmelts, to melt-viscosity-stable hotmelts, more particularly PU hotmelts, comprising these viscosity stabilizers, to a process for preparing them and to their use.

Reactive polyurethane hotmelts are a strongly growing product group within the applications of polyurethanes in the adhesives field. For their synthesis it is preferred to use linear polyester polyols and/or polyether polyols in combination with an excess of polyisocyanates, preferably diisocyanates.

The advantages of this product class lie above all in the absence of solvent, the possibility of applying the products under hot conditions at relatively low viscosities, while nevertheless obtaining high initial strengths and, after a relatively short time, by virtue of the further reaction with moisture, of obtaining adhesive bonds having a very high thermal stability, well above the application temperatures, and excellent solvent resistances.

Reactive hotmelt adhesive systems are usually provided in drums on the process line. In these drums, the hotmelt adhesive is then melted using—for example—special drum heaters. Depending on process and equipment, therefore, the reactive PU hotmelt systems experience considerable temperature loads which in some cases are long-lasting. This lasting temperature load has the disadvantageous effect of a significant rise in the melt viscosity of the hotmelt adhesive, and a decrease in the NCO content as a result of secondary reactions in which the remaining free NCO groups in the PU hotmelt system may be involved at elevated temperatures.

Since, moreover, reactive hotmelts are applied in the form of a melt, it must also be ensured at this stage that there is no increase in viscosity.

From the standpoint of the end user, therefore, there is a desire in principle for reactive hotmelt adhesive systems which possess excellent stabilities in viscosity under hot conditions.

DE-A 1 005 726 discloses carbodiimides as heat and water stabilizers, i.e. for protection against hydrolysis, for polyisocyanate-modified polyester compositions of homogeneous or porous structure. Here it has been found that the compression hardness in particular of polyurethane foams exhibits no drop or only a slight drop in the mechanical values on 12-day storage at 70° C./95% relative humidity as a result of the addition of tetramethylene-ω, ω′-bis-tert-butylcarbodiimide, in contrast to carbodiimide-free foams. DE-A 1 005 726, however, is concerned exclusively with the hydrolysis stability, which has no correlation with the viscosity stability at elevated temperature.

There is a reference to the effect of monomeric carbodiimides on the stability of the melt viscosity of isocyanate-terminated prepolymers under temperature load in Tappi Journal 1996, pp. 196-202. The monomeric carbodiimide compounds investigated are based on monomeric 4,4′-MDI and therefore possess free reactive NCO groups. It was found that the increase in melt viscosity on storage at 80° C. for 4 weeks relative to a 4,4′-MDI-based standard PU system rose less sharply for the system with monomeric carbodiimide. Furthermore, in the course of a 2-hour continuous-temperature loading of 121° C., the increase in viscosity of around 10%/h for the standard system was reduced to around 4%/h through use of monomeric carbodiimide. The usefulness of such systems on the industrial scale is limited, since they may exhibit outgasing and are not very resistant to migration.

The monomeric carbodiimide investigated in the aforementioned Tappi Journal, moreover, has free reactive NCO groups. Consequently, these compounds cannot be used as polyol stabilizers without a reaction with the polyol. Consequently the average molecular weight and, accordingly, the viscosity of the polyol component are increased. This is a disadvantage against the background of an optimum preparation process.

It was an object of the present invention, therefore, to provide hotmelt viscosity stabilizers which do not have the disadvantages of the prior art.

Surprisingly it has now been found that polymeric carbodiimides based on tetramethylenexylylene diisocyanate are outstandingly suitable as viscosity stabilizers.

The present invention accordingly provides new viscosity stabilizers for hotmelts, comprising at least one polymeric carbodiimide (A) based on tetramethylenexylylene diisocyanate.

Hotmelts for the purposes of the invention are all kinds of hotmelt adhesives, and PU hotmelts encompass all kinds of reactive hotmelt adhesives.

Carbodiimides (A) in the context of the present invention are substantially linear polycarbodiimides which have on average at least two carbodiimide groups per molecule.

The carbodiimide (A) is preferably an aromatic and/or aliphatic substituted aromatic polycarbodiimide.

The carbodiimide (A) for the purposes of the invention is preferably a compound of the formula

X₁—R—[—N═C═N—R—]ₘ—N═C═N—R—X₂,

in which R=

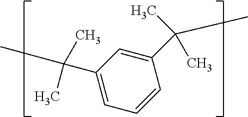

and
m has a value of at least 1, preferably 2-10, more preferably 3-5,
n has a value of at least 1, preferably 2 and 150, more preferably 2 and 75, very preferably 2 and 20,
X₁ and X₂ independently of one another are identical or different and are —N═C═O,

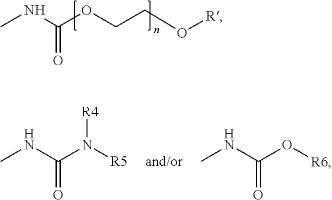

and
R′, R₄, R₅ and R₆=hydrogen, optionally substituted C₁-C₂₄ aliphatic, optionally substituted C₅-C₂₄ cycloaliphatic or optionally substituted C₆-C₂₄ aromatic hydrocarbon radicals.

It is further preferred for the viscosity stabilizer preferably to comprise carbodiimides (A) which possess no free and hence reactive isocyanate groups. These carbodiimides which possess no free and hence reactive isocyanate groups are prepared by the addition of a stoichiometric excess (relative to —NCO) of monoalcohols, such as polyethylene glycols (e.g. PEG550, PEG300). An OH number of 8 mg KOH/g to 20 mg KOH/g is preferred.

The aforementioned carbodiimides are commercially available components which are available, for example, from Rhein Chemie Rheinau GmbH, for example under the trade name Stabaxol® P 200, as a polymeric carbodiimide based on tetramethylenexylylene diisocyanate, optionally endcapped with monoalcohols, i.e. without free and hence reactive isocyanate groups.

The present invention further provides melt-viscosity-stable hotmelts which are characterized in that they comprise at least one polyester polyol and/or polyether polyol (B), at least one isocyanate (C) and at least one viscosity stabilizer (A) of the invention.

By the polyester/polyether polyols (B) are meant, in the context of the present invention, a polyol having more than one OH group, preferably two terminal OH groups. Polyols of this kind are known to the skilled person. Polyester polyols are preferred. They can be prepared by known routes, such as for example from aliphatic hydroxycarboxylic acids or from aliphatic and/or aromatic dicarboxylic acids and one or more diols. It is also possible to use appropriate derivatives, such as lactones, esters of lower alcohols or anhydrides, for example. Examples of suitable starting products are succinic acid, adipic acid, suberic acid, azelaic acid, sebacic acid, dodecanedioic acid, glutaric acid, glutaric anhydride, phthalic acid, isophthalic acid, terephthalic acid, phthalic anhydride, ethylene glycol, diethylene glycol, 1,4-butanediol, 1,6-hexanediol, neopentyl glycol and/or ε-caprolactone.

Polyester polyols at room temperature are either liquid (glass transition temperature Tg<20° C.) or solid. Polyester polyols which are solid at room temperature are either amorphous (glass transition temperature Tg>20° C.) or crystallizing.

Suitable crystallizing polyesters are those, for example, based on linear aliphatic dicarboxylic acids having at least 2 carbon atoms, preferably at least 6 carbon atoms, more preferably 6 to 14 carbon atoms in the molecule, such as adipic acid, azelaic acid, sebacic acid and dodecanedioic acid, for example, preferably adipic acid and/or dodecanedioic acid, and also on linear diols having at least 2 carbon atoms, preferably at least 4 carbon atoms, more preferably 4-6 carbon atoms in the molecule, preferably with an even number of carbon atoms, such as 1,4-butanediol and 1,6-hexanediol, for example. Also particularly suitable are the polycaprolactone derivatives, based on difunctional starter molecules, such as 1,6-hexanediol, for example.

Examples of suitable amorphous polyester polyols are those based on adipic acid, isophthalic acid, terephthalic acid, ethylene glycol, neopentyl glycol and/or 3-hydroxy-2,2-dimethylpropyl 3-hydroxy-2,2-dimethylpropanoate.

Examples of suitable polyester polyols which are liquid at room temperature are those based on adipic acid, ethylene glycol, 1,6-hexanediol and/or neopentyl glycol.

Suitable polyether polyols are the polyethers which are customary in polyurethane chemistry, such as, for example, the addition compounds or co-addition compounds of tetrahydrofuran, styrene oxide, ethylene oxide, propylene oxide, of the butylene oxides or of epichlorohydrin, preferably of ethylene oxide and/or of propylene oxide, that are prepared using difunctional to hexafunctional starter molecules, such as water, ethylene glycol, 1,2- or 1,3-propylene glycol, neopentyl glycol, glycerol, trimethylolpropane, pentaerythritol, sorbitol or amines having 1 to 4 NH bonds, for example. Preference is given to the difunctional propylene oxide adducts and/or ethylene oxide adducts and also to polytetrahydrofuran. Such polyether polyols and their preparation are known to the skilled person.

The aforementioned polyester and/or polyether polyols (B) are commercially available components, being available, for example, from BayerMaterial Science AG or from Evonik Degussa AG.

Examples of suitable isocyanate components C) are compounds having isocyanate contents of 5% to 60% by weight (based on the isocyanate) and having aliphatic, cycloaliphatic, araliphatic and/or aromatically attached isocyanate groups, such as, for example, 1,4-diisocyanatobutane, 1,6-diisocyanatohexane (HDI), 2-methyl-1,5-diisocyanatopentane, 1,5-diisocyanato-2,2-dimethylpentane, 2,2,4- and 2,4,4-trimethyl-1,6-diisocyanatohexane, 1,10-diisocyanatodecane, 1,3- and 1,4-diisocyanatocyclohexane, 1,3- and 1,4-bis(isocyanatomethyl)cyclohexane, 1-isocyanato-3,3,5-trimethyl-5-isocyanatomethylcyclohexane (isophorone diisocyanate, IPDI), 4,4′-diisocyanatodicyclohexylmethane, 1-isocyanato-1-methyl-4(3)isocyanatomethylcyclohexane, bis(isocyanatomethyl)norbornane, 1,3- and 1,4-bis(2-isocyanatoprop-2-yl)benzene (TMXDI), 2,4- and/or 2,6-diisocyanatotoluene (TDI), 2,2′-, 2,4′- and/or 4,4′-diisocyanatodiphenylmethane (MDI), 1,5-diisocyanatonaphthalene, 1,3- and 1,4-bis(isocyanatomethyl)benzene or mixtures of these. It will be appreciated that polyisocyanates can also be used.

Diisocyanates preferred as diisocyanate component C) are 1,6-diisocyanatohexane (HDI), 1-isocyanato-3,3,5-trimethyl-5-isocyanatomethylcyclohexane (isophorone diisocyanate, IPDI), 4,4′-diisocyanatodicyclohexylmethane, 2,4- and/or 2,6-diisocyanatotoluene (TDI), 2,2′-, 2,4′- and/or 4,4′-diisocyanatodiphenylmethane (MDI).

Diisocyanates particularly preferred as diisocyanate component C) are 2,4′- and/or 4,4′-diisocyanatodiphenylmethane (MDI).

The aforementioned isocyanates (C) are commercially available components, being available, for example, from BayerMaterial Science AG under the trade name Desmodur® 44 M.

The melt-viscosity-stable hotmelts of the invention preferably have a molar ratio of isocyanate (NCO)C) to polyester polyol and/or polyether polyol (OH) B) of >1 and carbodiimide concentrations (A), based on the polyol (B), of 0.05% by weight to 10% by weight.

The melt-viscosity-stable hotmelts of the invention may also be provided with further additives.

In another embodiment of the invention, the melt-viscosity-stable hotmelts comprise, as further additives, catalysts which are activating with moisture, organic or inorganic fillers, colorants, antioxidants, resins, reactive or unreactive polymers and/or extender oils.

The present invention further provides a process for preparing the melt-viscosity-stable hotmelts of the invention, whereby the viscosity stabilizer (A) is added to at least one polyester and/or polyether (B) and/or at least one isocyanate (C) or to the reaction product of at least one polyester and/or polyether (B) and at least one isocyanate (C).

In one preferred embodiment of the present invention, the melt-viscosity-stable hotmelts are obtainable by reaction of
at least one C₆-C₂₄ aromatic, C₁-C₂₄ aliphatic, C₇-C₂₄ araliphatic and/or C₅-C₂₄ cycloaliphatic diisocyanate (C), preferably having a free NCO group content of 5 to 60% by weight, more preferably of 20% to 55% by weight, very preferably of 30% to 50% by weight (based on (C))
and
a polyester polyol and/or polyether polyol (B) comprising the viscosity stabilizer of the invention (carbodiimide) (A), obtainable by
a) mixing of the polyester polyol and/or polyether polyol (B) with the viscosity stabilizer of the invention (carbodiimide) (A) at temperatures from 0° C. to 200° C., preferably 25° C. to 150° C., more preferably 80° C. to 120° C., with carbodiimide concentrations, based on the polyester polyol and/or polyether polyol (B), of 0.05% by weight to 10% by weight, preferably 0.5% by weight to 5% by weight, more preferably 0.8% by weight to 3.5% by weight, in a mixing time of 0.1 min to 240 min, preferably 1 min to 180 min, more preferably 5 min to 120 min,
   the ratio of C to B being selected such that the molar ratio of NCO to OH is >1, preferably from 1.2 to 4.0, more preferably from 1.3 to 3.0.

The reactive polyurethane systems and/or preparations of the invention are prepared, for example, by first mixing the polyester polyol and/or polyether polyol B) with the viscosity stabilizer of the invention (carbodiimide) A), with stirring, at temperatures from 0° C. to 200° C., preferably 25° C. to 150° C., more preferably 80° C. to 120° C., with carbodiimide concentrations, based on the polyol, of 0.05% by weight to 10% by weight, preferably 0.5% by weight to 5% by weight, more preferably 0.8% by weight to 3.5% by weight, in a mixing time of 0.1 min to 240 min, preferably 1 min to 180 min, more preferably 5 min to 120 min, and then mixing that mixture with an excess of polyisocyanates C), the ratio of (C) to (B) being selected such that the molar ratio of NCO to OH is >1, preferably from 1.2 to 4.0, more preferably from 1.3 to 3.0, and dispensing the homogeneous mixture or else stirring it until a constant NCO value is obtained and then dispensing it. The reaction temperature selected is 60 to 150° C., preferably 80 to 130° C.

It will be appreciated that the reactive polyurethane systems and/or preparations may also be prepared continuously in a stirred-tank cascade or in suitable mixing assemblies, such as high-speed mixers operating on the rotor-stator principle, or a static mixer, for example.

It is of course possible to modify the polyester polyols and/or polyether polyols or a part thereof with a substoichiometric amount of diisocyanates (C), preferably 1,6-disocyanatohexane (HDI), 2,4- and/or 2,6-diisocyanatotoluene (TDI) and/or 2,4′- and/or 4,4′-diisocyanatodiphenylmethane (MDI), and, after the end of reaction, to react the urethane-group-containing polyols with an excess of diisocyanates to form a hotmelt containing isocyanate groups.

It is also possible to carry out the reaction of the polyester polyol and/or polyether polyol (B) with the diisocyanates (C) in the presence of up to 5% by weight of, for example, trimers of aliphatic diisocyanates, such as HDI, for example, or to add such trimers after the end of prepolymerization.

The present invention further provides, moreover, for the use of the viscosity stabilizers of the invention in adhesives.

The present invention further provides, moreover, for the use of the melt-viscosity-stable hotmelts of the invention as a sealant, as a foam, as an adhesive, particularly for coating, as a hotmelt adhesive, as an assembly adhesive for provisional fixing of components, as a bookbinding adhesive, as an adhesive for producing cross-bottom valve bags, for producing composite films and laminates, as a laminating adhesive or as an edgebanding adhesive, and for coating.

The improvement in the stability of the melt viscosity at elevated temperature through the addition of carbodiimides (A) is demonstrated using the examples below, without the invention being restricted to the examples.

### WORKING EXAMPLES

Unless noted otherwise, all percentages are by weight.

The inventive and comparative examples used the following raw materials:

### Carbodiimide (A):

Stabaxol® P 200, a polymeric endcapped carbodiimide based on tetramethylenexylylene diisocyanate, Rhein Chemie Rheinau GmbH, Mannheim, Del.

### Polyester Polyol (B):

A polyester polyol based on adipic acid and 1,6-hexanediol was used, having a hydroxyl number of about 30 mg KOH/g and an acid number of about 0.5 mg KOH/g, obtained from Bayer MaterialScience AG with the trade name Baycoll® AD5027.

### Isocyanate I (C):

Desmodur® 44M (4,4′-diphenylmethane diisocyanate), available from Bayer MaterialScience AG.

### Preparation of the Reactive Polyurethane Hotmelts

### Inventive and Comparative Examples

The fractions of polyester polyol indicated in Table 1 are introduced into a 2-litre flat-flange beaker and are melted at 130° C. and then dewatered for 1 h at 130° C. under an underpressure of 15 mbar (+/−10 mbar). The dewatered polyester polyol is then admixed with the corresponding amount of carbodiimide under the conditions apparent from Table 1. Thereafter the corresponding molar amount of isocyanate I is added. After a stirred-incorporation time of 20 minutes, the products are dispensed into aluminium cartridges, which are given an airtight seal. The cartridges are then conditioned in a forced-air drying cabinet at 100° C. for 4 hours.

TABLE 1
Composition of the inventive and comparative examples

| | | Inventive | Inventive | Comparative |
|---|---|---|---|---|
| Identification | | Example 1 | Example 2 | Example 1 |
| Polyester polyol | [% by | 83.4 | 84.8 | 85.5 |
| | weight] | | | |
| Carbodiimide | [% by | 0.7 | 0.7 | 0.0 |
| Stabaxol ® P 200 | weight] | | | |
| Exposure time | [min.] | 120 | 5 | — |
| Exposure temperature | [° C.] | 80 | 80 | — |
| Concentration of | [% by | 0.83 | 0.83 | 0.0 |
| carbodiimide C in the | weight] | | | |
| polyester polyol | | | | |
| Isocyanate I | [% by | 14.8 | 14.5 | 14.5 |
| | weight] | | | |
| NCO content | [% by | 3.0 | 3.0 | 3.0 |
| (theoretical) | weight] | | | |
| Index | | 2.51 | 2.62 | 2.61 |

### Characterization of the Reactive Polyurethane Hotmelts:

Prior to investigation, the products dispensed into aluminium cartridges are melted in a forced-air heating cabinet at approximately 125° C. for approximately 30 minutes. The viscoelastic properties of the reactive polyurethane hotmelts are characterized using the MCR 301 rheometer from Anton-Paar. The spindle/measuring-cup system Z4 and CC27 was used. The viscosity was recorded as a function of shear rate and was evaluated using the Carreau-Yasuda algorithm. The accelerated viscosity test was carried out on the same instrument. This was done by measuring the sample at 120° C. for 2 hours (2 h test). The zero-point viscosity was determined by extrapolation and used for calculation of the increase in viscosity in %/h.

The NCO content was determined in accordance with DIN EN 1242.

### Determination of the Hydrolysis Stability of the Reactive Polyurethane Hotmelts:

The hotmelts of Inventive Examples 1-2 and of Comparative Examples 1 and 2 are cured as a film having a film thickness as defined in accordance with DIN EN ISO 527-1.3, for 14 days under standard conditions. S2 test specimens are punched from this cured film and are stored underwater at 60° C. for 24 hours. Following removal, the test specimens are reconditioned under standard conditions for 24 hours. The samples are then stored at 87° C. and 95% relative humidity for 0, 3, 5, 8, 14, 19, 27 and 31 days. The mechanical strength of the samples is determined via tensile testing in accordance with DIN EN ISO 527-1.3.

The results obtained in these tests are listed in Table 2.

TABLE 2
Rheological and mechanical properties of the inventive and comparative
examples

| | | Inventive | Inventive | Comparative |
|---|---|---|---|---|
| Identification | | Example 1 | Example 2 | Example I 1 |
| Viscosity at | | | | |
| 100° C. | [mPa * s] | 7498 | 7428 | 8032 |
| 130° C. | [mPa * s] | 3117 | 3144 | 3342 |
| Viscosity increase | [%/h] | 4.7 | 6.1 | 10.0 |
| (2 h test) | | | | |
| Tensile strengths | | | | |
| after 87° C./95% rel. | | | | |
| humid. | | | | |
| 0 days | [MPa] | 38 | 37 | 34 |
| 3 days | [MPa] | 37 | 36 | 35 |
| 5 days | [MPa] | 37 | 35 | 25 |
| 8 days | [MPa] | 37 | 32 | 14 |
| 14 days | [MPa] | 16 | 32 | 0 |
| 19 days | [MPa] | 10 | 32 | 0 |
| 27 days | [MPa] | 0 | 13 | 0 |
| 31 days | [MPa] | 0 | 11 | 0 |

### Discussion of Results:

Table 2 shows that the hydrolysis stability of Inventive Examples 1 to 2 can be improved significantly by using carbodiimide based on tetramethylenexylylene diisocyanate in comparison to the systems of the prior art (Comparative Example 1).

It is further apparent that the stability of the melt viscosity of Inventive Examples 1 to 2 can be improved likewise significantly by using a polymeric carbodiimide based on tetramethylenexylylene diisocyanate in comparison to system of the prior art without carbodiimide (Comparative Example 1). Hence the increase in melt viscosity can be reduced by using, for example, 0.83% by weight of Stabaxol® P200 (Inventive Example 1), relative to the system of the prior art (Comparative Example 1), by 5.3%/h.

What is claimed is:

1. Viscosity stabilizer for hotmelts, comprising at least one polymeric carbodiimide based on tetramethylenexylylene diisocyanate (A).

2. Viscosity stabilizer according to claim 1, characterized in that the polymeric carbodiimide (A) is an aromatic or aliphatic substituted aromatic polycarbodiimide.

3. Viscosity stabilizer according to claim 2, characterized in that the carbodiimide (A) is a compound of the formula
X₁—R—[—N═C═N—R—]ₘ—N═C═N—R—X₂
in which R= 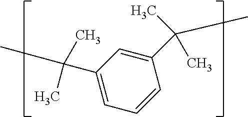
and
m has a value of at least 1,
n has a value of at least 1,
X₁ and X₂ independently of one another are identical or different and are —N═C═O, 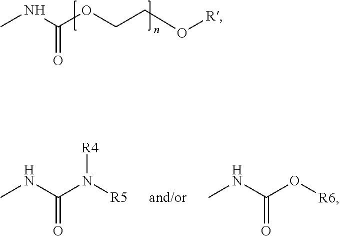
and
R′, R₄, R₅, R₆=hydrogen, optionally substituted C₁-C₂₄ aliphatic, optionally substituted C₅-C₂₄ cycloaliphatic or optionally substituted C₆-C₂₄ aromatic hydrocarbon radicals.

4. Viscosity stabilizer according to claim 1, characterized in that it is composed of carbodiimides (A) which possess no free reactive isocyanate groups.

5. Melt-viscosity-stable hotmelts characterized in that they comprise at least one polyester polyol and/or polyether polyol (B), at least one isocyanate (C) and at least one viscosity stabilizer according to claim 1.

6. Melt-viscosity-stable hotmelts according to claim 5, characterized in that they have a molar ratio of isocyanate(NCO) C) to the polyester polyol and/or polyether polyol(OH) B) of >1 and carbodiimide concentrations, based on the polyol, of 0.05% by weight to 10% by weight.

7. Melt-viscosity-stable hotmelts according to claim 5, characterized in that they comprise, as further additives, catalysts which activate with moisture, organic or inorganic fillers, colorants, antioxidants, resins, reactive or unreactive polymers and/or extender oils.

8. Process for preparing melt-viscosity-stable hotmelts according to claim 6, characterized in that the viscosity stabilizer is added to at least one polyester and/or polyether and/or at least one isocyanate or to the reaction product of at least one polyester and/or polyether and at least one isocyanate.

9. Process for stabilizing the viscosity in adhesives by adding at least one polymeric carbodiimide based on tetramethylenexylylene diisocyanate (A) according to claim 1.

10. Process for the production of coatings, sealants, foams, adhesives, composite films and laminates by adding hotmelts according to claim 5.
